(19) United States
(12) Patent Application Publication
(10) Pub. No.: US 2021/0205187 A1
(43) Pub. Date: Jul. 8, 2021

(54) Process For Forming A Deodorant

(71) Applicant: Lark Isely, Waitsburg, WA (US)

(72) Inventor: Lark Isely, Waitsburg, WA (US)

(21) Appl. No.: 17/206,700

(22) Filed: Mar. 19, 2021

(51) Int. Cl. A61K 8/19; A61K 8/9789; A61Q 15/00

(52) U.S. Cl. CPC A61K 8/19; A61Q 15/00; A61K 8/9789

(57) ABSTRACT

A method for forming a deodorant, wherein the method comprises: providing a raw material containing mgcl2; dissolving the material in heated h2o to a solute content close to saturated to form a solution; determining a molar amount of mgcl2 dissolved; dissolving a caustic hydroxide in the h2o to a ratio 5% less than what will react with the mgcl2 to a near saturated solution; precipitating mgoh2 from the solution to form a mass and removing surfaced brine water; removing excess h2o from the mass; measuring the mass for quantities of mgoh2, cl mineral salts and h2o; and processing the mass with a specifically super-fatted saponified coconut oil.

### TECHNICAL FIELD

This invention relates to methods and processes for forming deodorants for humans.

### BACKGROUND OF THE INVENTION

The subject invention generally relates to a method and processes for forming deodorants for humans.

### BRIEF SUMMARY OF EMBODIMENTS OF THE INVENTION

According to one embodiment of the invention a method for forming a deodorant, wherein the method comprises: providing a raw material containing mgcl2; dissolving the material in heated h2o to a solute content close to saturated to form a solution; determining a molar amount of mgcl2 dissolved; dissolving a caustic hydroxide in the h2o to a ratio 5% less than what will react with the mgcl2 to a near saturated solution; precipitating mgoh2 from the solution to form a mass and removing surfaced brine water; removing excess h2o from the mass; measuring the mass for quantities of mgoh2, cl mineral salts and h2o; and processing the mass with a specifically super-fatted saponified coconut oil.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

FIG. 1 is a table of parameters for forming different embodiments of the deodorant for human use.

### DETAILED DESCRIPTION OF THE EMBODIMENTS OF THE INVENTION

The present invention is a method of forming a deodorant for human use in combating foul odors emitted from the human body. The method comprises a multi-phase process comprising three parts. Part 1. Create a mineral dense hydrated gelatinous mass. Part 2. Create a surfactant product by partially saponifying fatty acids. Part 3. Measure additional components, from a recipe, of base oils, butters, waxes, powders, and essential oils. These additional components are added in a rated process to the above mentioned components and mixed at specific temperatures and mixing power with a micro-emulsion mixing process at specific temperatures. The final product must be put into consumer containers at a specific temperature and sufficiently cooled before capping.

The method starts with obtaining any raw material with a high content of MgCl2, including pure MgCl2. Next the mineral material is dissolved in heated* H2O to a specific solute content close to saturated. Optionally, liquid brine with sufficient MgCl2 can be used. The molar measurement of mineral is calculated with a working knowledge of the molar mass of the MgCl2. Based on the molar mass of the MgCl2, a concentration of a caustic hydroxide is determined. The hydroxide is dissolved in H2O to a ratio 5% less than what will react with the MgCl2 to a near saturated solution. This process is carried out at specific temperatures and timeframes. *H2O temperature range is 140 degrees F. to boiling. #H20 temperature range is from 60 to 80 degrees F.

There is a specific static pot time to allow the resultant product with a high concentration of MgOH2 to precipitate and settle to the bottom of the pot. While MgOH2 precipitates, surfaced brine water is removed. Pot time is 24 to 72 hours.

After the pot time of the product, the mass is moved to a vacuum process where excess H2O is removed from the product. The final product is measured to contain specified quantities of MgOH2, Cl mineral salts and H2O depending on the desired finished product. This water containing mass is then processed with a specifically super-fatted saponified coconut oil and may include other oils as well. he resulting product is a dispersed liquid-like mass of the hydrated MgOH2 made from the first process. The product is now ready to mix with specific recipes of oils, other solids, and/or waxes to create a consumer friendly deodorant.

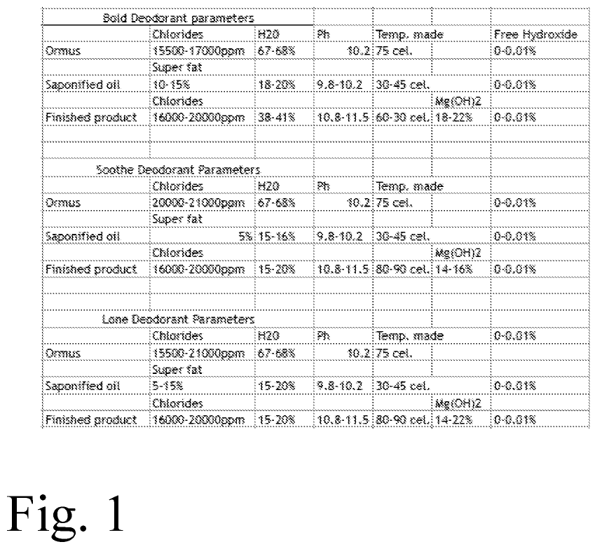

FIG. 1 lists specific parameters of concentrations of solids, fats, and water plus temperatures needed during the processes for forming various embodiments of the deodorant in accordance with the described method.

What is claimed is:

1. A method for forming a deodorant, wherein the method comprises:
providing a raw material containing MgCl2;
dissolving the material in heated H2O to a solute content close to saturated to form a solution;
determining a molar amount of MgCl2 dissolved;
dissolving a caustic hydroxide in the H2O to a ratio 5% less than what will react with the MgCl2 to a near saturated solution;
precipitating MgOH2 from the solution to form a mass and removing surfaced brine water;
removing excess H2O from the mass;
measuring the mass for quantities of MgOH2, Cl mineral salts and H2O; and
processing the mass with a specifically super-fatted saponified coconut oil.

2. The method of claim 1 wherein the mass is mixed with one of oils, solids, and waxes.
